(19) United States
(12) Patent Application Publication
(10) Pub. No.: US 2022/0088311 A1
(43) Pub. Date: Mar. 24, 2022

(54) Multi-Function Syringe

(71) Applicant: Hubertus Goller Gesellschaft m.b.H., Wien (AT)

(72) Inventor: Patrick Gruber, Wien (AT)

(21) Appl. No.: 17/447,751

(22) Filed: Sep. 15, 2021

(30) Foreign Application Priority Data
Sep. 23, 2020 (EP) 20197755.0

(51) Int. Cl. A61M 5/31

(52) U.S. Cl. CPC A61M 5/3134; A61M 2005/3142; A61M 5/31

(57) ABSTRACT

The invention relates to a multi-function syringe, comprising a syringe barrel and a syringe plunger axially displaceable within the syringe barrel, whereby there is formed an receiving volume enclosed by the syringe barrel and the syringe plunger, wherein the syringe barrel has a syringe outlet integrally formed thereon for discharging a fluid present in the receiving volume, wherein the syringe barrel has at the transition to the syringe outlet a cylindrical platform, which has a smaller diameter than a body of the syringe barrel and a larger diameter than the syringe outlet, wherein at the syringe outlet there may be mounted a bladder syringe attachment having a break-off tip for using the multi-function syringe as a bladder syringe.

The invention relates to a multi-function syringe, comprising a syringe barrel and a syringe plunger axially displaceable within the syringe barrel, whereby there is formed a receiving volume enclosed by the syringe barrel and the syringe plunger, whereby the syringe barrel has a syringe outlet integrally formed thereon for discharging a fluid present in the receiving volume.

From prior art there have been known standard syringes, in which a syringe plunger, respectively, is arranged axially displaceable within the syringe barrel. As a standard, these syringes have a syringe outlet, which is configured according to the standard ISO-80369-7 as a Luer cone. In this way, the syringe is compatible with other standardized connections.

Another syringe known from prior art is marketed by Cathejell as a bladder syringe having a break-off tip. This syringe is an accordion syringe, i.e. it is configured without plunger and having a compressible syringe barrel. It has been known that the accordion syringe is filled with a lubricant and a bladder syringe attachment is applied onto an open end of the accordion syringe for closing the accordion syringe. The bladder syringe attachment is closed by the break-off tip and may be manually opened by manually breaking off the break-off tip from the bladder syringe attachment. The break-off tip is attached within the bladder syringe attachment in a way such that this has no sharp edges after breaking off the break-off tip.

The mentioned bladder syringe by Cathejell is configured such that the syringe body comprises, on the one hand, the compressible accordion section and, on the other hand, a rigid and disc-like front section spaced apart from the accordion section, which is enclosed by the bladder syringe attachment. Since a disc-like front section is not present with standard syringes, it is not possible to directly attach a bladder syringe attachment onto standard syringes.

It is the task of the invention to reconfigure a commercially available syringe in a constructive way using a syringe barrel and a syringe plunger axially displaceable within the syringe barrel such that this may be used for a plurality of purposes, in particular as a bladder syringe. If, for example, the same syringe barrels may be used for several different types of syringes, then mass production of syringe barrels may be simplified.

This task is solved by a multi-function syringe, comprising a syringe barrel and a syringe plunger axially displaceable within the syringe barrel, whereby there is formed a receiving volume enclosed by the syringe barrel and the syringe plunger, whereby the syringe barrel has a syringe outlet integrally formed thereon for discharging a fluid present in the receiving volume, wherein the syringe barrel has at the transition to the syringe outlet a cylindrical platform, which has a smaller diameter than a body of the syringe barrel and a larger diameter than the syringe outlet, wherein at the syringe outlet there may be mounted a bladder syringe attachment having a break-off tip for using the multi-function syringe as a bladder syringe.

With the multi-function syringe according to the invention, there is provided a platform for attaching a bladder syringe attachment in addition to the syringe outlet—and not alternatively thereto. In this way, the multi-function syringe may be produced as a unit component independently of the further purpose of use, whereby mass production of a single component for bladder syringes as well as for syringes closed by caps is being enabled. A further advantage of the multi-function syringe according to the invention is that the syringe may be continued to be filled from the front through the syringe outlet due to the bladder syringe attachment that may be later on mounted. This is not possible with an integrally configured syringe having a break-off tip. Such a syringe may only be filled from the rear side, and the seal and the plunger rod of the syringe would have to be mounted later on in a more cumbersome way. The configuration of seal and plunger rod for a filling from the rear side is also in general more expensive.

The platform according to the invention between syringe barrel and syringe outlet, hence, provides for the attachment of diverse non-standardized closures such as, in particular, a bladder syringe attachment having a break-off tip. This solution is insofar special as there is further given the possibility to close the syringe outlet using other auxiliary means such as a cap in order to provide optionally to the closing means having a bladder syringe attachment also a closing means having a removable cap. In this way, in mass production there may be manufactured a single syringe body for a plurality of purposes of use. The challenge of having to manufacture different syringe bodies for different application purposes, consequently, is omitted.

The inventive solution of the platform between syringe barrel and syringe outlet furthermore has the advantage that the external diameter of the syringe barrel is not increased even with a bladder syringe attachment applied, as it is the case with the bladder syringe by Cathejell.

With the solution according to the invention, there is also provided a plunger syringe for the possible use as a bladder syringe having a standard external diameter.

It is especially preferred if the syringe outlet has a length and a tip diameter, which deviate from the standard dimensions of a Luer cone standardized in the ISO-80369-7, wherein the length of the syringe outlet is preferably larger than the length of a Luer cone standardized in the ISO-80369-7 and the tip diameter of the syringe outlet is larger than the tip diameter of a Luer cone standardized in the ISO-80369-7. In this way, it is prevented that the syringe may be accidently used by the user for a standardized Luer connection. According to any alternative embodiment variant, the syringe outlet may be configured as a Luer cone according to ISO-80369-7. Furthermore, only the syringe outlet or only the tip diameter ds may deviate from the standard dimensions of a Luer cone standardized in the ISO-80369-7.

It is further preferred that the syringe barrel is an integrally manufactured injection-moulded part. Thereby, on the one hand the production of the syringe barrel is simplified, and on the other hand the multi-function syringe is also less prone to failures. Releasable embodiments, wherein the platform is applied later on onto an already existent standard syringe, are thus excluded.

In an especially advantageous embodiment the platform mentioned has at the external circumference thereof a rib for attachment of the bladder syringe attachment. The rib makes it possible to mount the bladder syringe attachment onto the syringe barrel without using any further auxiliary means. In this way, there is provided an especially fast closure of the multi-function syringe using the bladder syringe attachment.

If the bladder syringe attachment is applied onto the multi-function syringe, then there is provided according to the invention a bladder syringe. In order to be compatible with the multi-function syringe, the bladder syringe attachment used has a mounting section facing away from the break-off tip and having an internal diameter, which essentially corresponds to the external diameter of the platform of the multi-function syringe.

In order to attach the bladder syringe attachment onto the multi-function syringe in a stable and preferably also permanent way, there may be used the two embodiment variants described in the following, which may also be combined.

In the first embodiment variant the mounting section of the bladder syringe attachment may be non-releasably mounted onto the platform, preferably by ultra-sound welding, laser welding or bonding. In this solution there are used additional auxiliary means for attachment in order to connect the bladder syringe attachment in an especially solid and permanent way to the multi-function syringe.

In the second embodiment variant the mounting section of the bladder syringe attachment has a groove on the internal circumference thereof for engagement with the rib of the platform such that the rib may be brought into engagement with the groove. The releasability or non-releasability, respectively, of the rib/groove connection may be affected by the choice in regard to materials, shapes and dimensions. In addition, there may be provided by the shape of the rib or groove, respectively, that only a particular bladder syringe attachment having a predefined groove may be applied onto the multi-function syringe.

The multi-function syringe and the bladder syringe attachment are further preferably composed of the same material, which is advantageous in particular when the application of the bladder syringe attachment onto the multi-function syringe is realized by welding.

In order to achieve a compact configuration of the bladder syringe, there may be additionally provided that the mounting section of the bladder syringe attachment has an external diameter, which corresponds to the diameter of the syringe barrel at most.

In order to further improve the bladder syringe, the bladder syringe attachment may have an internal profile, which corresponds essentially to the external profile of the syringe outlet, and wherein the external profile of the syringe outlet will rest on the internal profile of the bladder syringe attachment if the bladder syringe attachment is mounted on the multi-function syringe. In this way, the bladder syringe attachment may be applied press-fit onto the syringe outlet, such that a wiggling of the bladder syringe attachment may be prevented. Another advantage is that in this way no medium present in the multi-function syringe may enter the gaps or cavities of the contours of the inventive multi-function syringe and remain within the multi-function syringe in use as a residual amount unused.

If the multi-function syringe is not to be used as a bladder syringe, this may also be closed by means of a general cap, which does not interact with the platform mentioned. In this variant the syringe is designated as a multi-closure syringe and comprises the multi-function syringe in one of the embodiments mentioned above as well as a cap, which has a smaller length than the syringe outlet and releasably closes the syringe outlet. It is in particular provided that the multi-closure syringe is marketed as a wound gel syringe, i.e. the multi-closure syringe is filled with, for example, wound gel, before it is closed using the cap.

In a further aspect the multi-function syringe may be provided in combination with a bladder syringe attachment not yet mounted and/or a cap not yet mounted as kit-of-parts. In this way, there may be realized a reconfiguration of the syringe into a bladder syringe or multi-closure syringe quickly before marketing.

Advantageous and non-limiting embodiments of the invention are explained in the following by way of the drawings.

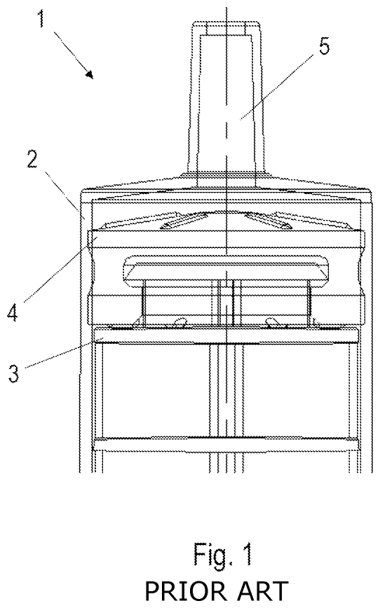

FIG. 1 shows a standard syringe according to prior art.

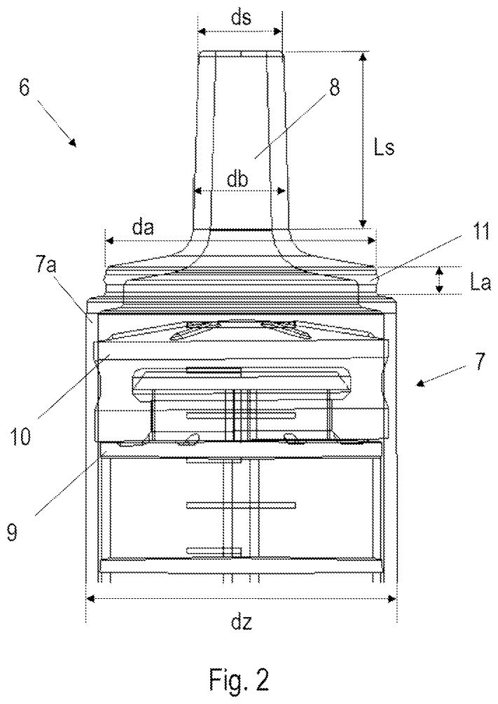

FIG. 2 shows a detail of an inventive multi-function syringe.

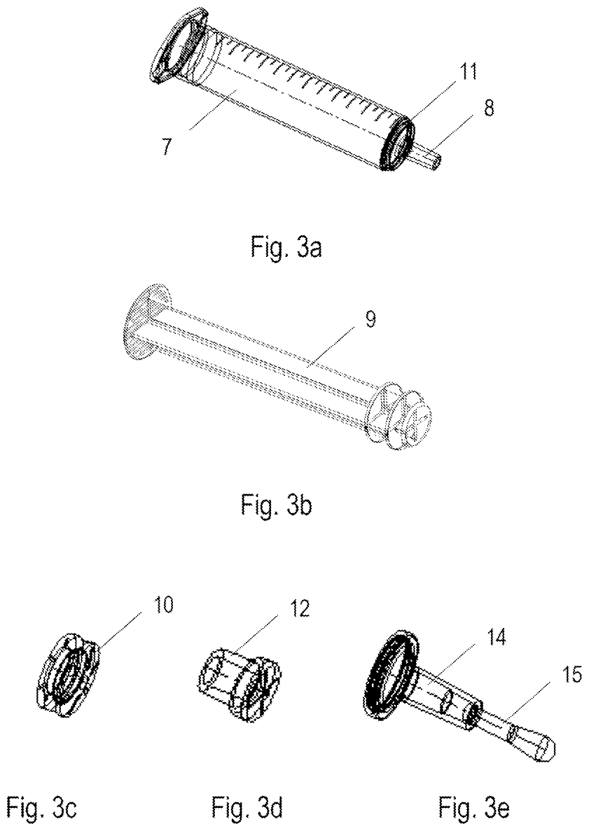

The FIGS. 3a to 3c show the components of the multi-function syringe of FIG. 2.

The FIGS. 3d and 3e show two different closure embodiments for the multi-function syringe of FIG. 2.

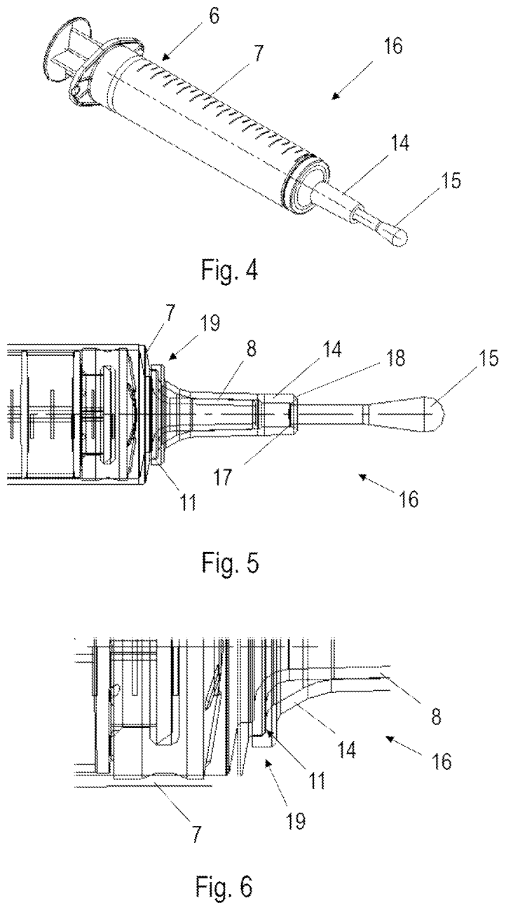

FIG. 4 shows a multi-function syringe configured as a bladder syringe.

The FIGS. 5 and 6 each show a detail of a first embodiment variant of a bladder syringe.

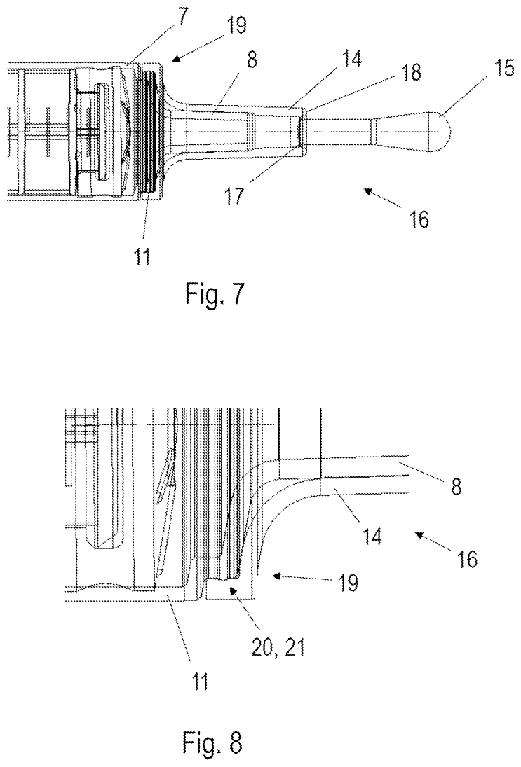

The FIGS. 7 and 8 each show a detail of a second embodiment variant of a bladder syringe.

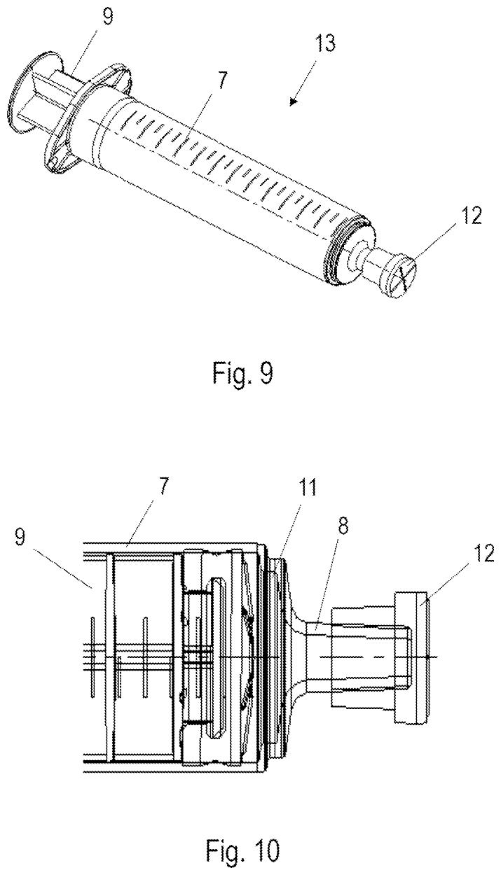

FIG. 9 shows a multi-function syringe configured as a multi-closure syringe.

FIG. 10 shows a detail of the multi-closure syringe of FIG. 9.

FIG. 1 shows a standard syringe 1 according to prior art. The standard syringe 1 is configured as a plunger syringe and comprises a syringe barrel 2 and a syringe plunger 3 axially displaceable within the syringe barrel 2. The syringe plunger 3 is sealed against the syringe barrel 2 by means of a sealing element 4. If the syringe plunger 3 is pulled out of the syringe barrel 2, there is formed a receiving volume not further depicted for receiving a fluid between the syringe barrel 2 and the syringe plunger 3. In an embodiment known, the fluid received in the receiving volume is, for example, a wound gel. Further fluids used in standard syringes 1 according to prior art are known to those skilled in the art.

For receiving and discharging the fluid into or out of, respectively, the receiving volume, the syringe barrel 2 has a syringe outlet 5, which is configured as a Luer cone standardized according to the ISO-80369-7. The syringe outlet 5 may be closed using a compatible cap.

FIG. 2 shows a multi-function syringe 6 according to the invention. Also the multi-function syringe 6 comprises a syringe barrel 7 (FIG. 3a) having a syringe outlet 8 and a syringe plunger 9 axially displaceable within the syringe barrel 7 (FIG. 3b), which may be sealed against the syringe barrel 7 by means of a sealing element 10 (FIG. 3c). Analogously to the standard syringe 1 of FIG. 1, the syringe plunger 9 may be pulled out of the syringe barrel 8 such that there is formed a receiving volume not further depicted for receiving a fluid between the syringe barrel 7 and the syringe plunger 9.

Whereas the syringe plunger 9 and the sealing element 10 may be configured according to prior art, i.e. as in FIG. 1, the syringe barrel 7 of the multi-function syringe 6 differs from the syringe barrel 2 of the standard syringe 1. In contrast to the embodiment of FIG. 1, the syringe barrel 7 of the multi-function syringe 6 of FIG. 2 has a platform 11 at the transition to the syringe outlet 8, i.e., the platform 11 is situated between a body 7a of the syringe barrel 7 and the syringe outlet 8 of the syringe barrel 7. Body 7a of the syringe barrel 7 is designated as the area in which the syringe plunger 9 is accommodated.

From FIG. 2 there is, in addition, to be seen that the body 7a of the syringe barrel 7 has an external diameter dz, which is larger than the external diameter da of the platform 11. The syringe outlet 8 is conical in this embodiment and has a basis diameter db facing the platform 11 and a tip diameter ds facing away from the platform 11 and being smaller than the basis diameter db. This results in proportions of dz>da>db>ds. In general, the basis diameter db is herein also designated as diameter of the syringe outlet 8, even if the syringe outlet 8 is conical. The multi-function syringe 6 further has a length Ls of the syringe outlet and a height La of the platform 11.

According to an exemplary embodiment of the multi-function syringe 6 according to the invention having a volume of 10 ml, the multi-function syringe 6 has the following dimensions:

External diameter dz=17.24 mm

External diameter da=15.00 mm

Basis diameter db=5.31 mm

Tip diameter ds=4.72 mm

Length Ls=9.90 mm

Height La=1.60 mm

Depending on the embodiment, the syringe outlet 8 may be configured as a Luer cone, i.e. it may have a length Ls and a tip diameter ds, as standardized in the ISO-80369-7. In the multi-function syringe 6 described herein there is in general provided that the syringe outlet 8 has a length Ls and a tip diameter ds, which deviate from the standard dimensions of a Luer cone standardized in the ISO-80369-7. The length Ls of the syringe outlet 8 may, for example, be larger than the length of a Luer cone standardized in the ISO-80369-7, and the tip diameter ds of the syringe outlet 8 may be larger than the tip diameter of a Luer cone standardized in the ISO-80369-7.

According to an alternative embodiment variant, also only the length Ls of the syringe outlet 8 or only the tip diameter ds may deviate from the standard dimensions of a Luer cone standardized in the ISO-80369-7.

Tip diameter ds and basis diameter db as well as the length Ls are configured, depending on an embodiment, as a Luer cone according to ISO-80369-7, or, as explained above, as a cone, which is larger than a Luer cone and may thus not accidentally be used with a standardized Luer connection.

The external diameter dz depends on the volume of the multi-function syringe 6 and may be smaller or larger than stated above.

According to the volume of the multi-function syringe 6 and, hence, depending on the external diameter dz or the embodiment of the syringe outlet 8 and the basis diameter db, respectively, the external diameter da for the platform 11 has to be adjusted accordingly.

The height La of the platform 11 is also adjusted to the volume of the inventive multi-function syringe 6. This is realized in order to enable a solid mechanical connection corresponding to the size of the bladder syringe attachment 14 to be mounted. The height La is preferably in a range of between 1 mm and 5 mm.

The multi-function syringe 6 may be used for various fields of use due to the particular embodiment thereof having a platform 11 and a syringe outlet 8 present nevertheless. Thus, FIG. 3d shows a cap 12, which may be applied upon the syringe outlet 8. The cap 12 may usually be removed from the syringe outlet 8 and then be reapplied such that the multi-function syringe 6 may be reclosed. For this reason, the syringe herein may be designated as a multi-closure syringe 13 and may, for example, be used as a wound gel syringe. This use is described in greater detail below by way of the FIGS. 9 and 10.

FIG. 3e shows a bladder syringe attachment 14 having a break-off tip 15. As is explained in the following by way of the FIGS. 4 to 8, the multi-function syringe 6 having a bladder syringe attachment 14 applied may be used as a bladder syringe 16. The bladder syringe attachment 14 has the advantageous characteristic that the break-off tip 15 may be broken off using only one hand if the bladder syringe 16 is picked up with one hand and the break-off tip 15 is pressed onto an unyielding surface. In this way, the bladder syringe 16 may be opened especially hygienically. Another advantage is that the bladder syringe 16 according to the invention may be opened in this way using only one hand.

Anticipating the FIGS. 5 and 7, it is further obvious that the break-off tip 15, before breaking within the bladder syringe attachment 14, is attached to a predetermined breaking point 17 such that, after breaking, there will not be present any external breaking edge. In this way, the opening 18 of the bladder syringe attachment 14 may be guided, after breaking off the break-off tip 15, for example, into or to the urinary tract, without any damage thereto.

FIG. 4 shows the bladder syringe 16, comprising the multi-function syringe 6 and the bladder syringe attachment 14 having the break-off tip 15. It is obvious that the syringe outlet 8 is accommodated fully within the bladder syringe attachment 14. FIG. 5 shows in detail that the bladder syringe attachment 14 may have an internal contour, which essentially corresponds to the external contour of the syringe outlet 8. In this way, the bladder syringe attachment 14 may be applied onto the syringe outlet 8 without any clearance.

It is obvious from FIG. 6 that the platform 11 has an essentially cylindrical form with a smooth surface. The bladder syringe attachment 14 has at the end facing away from the break-off tip a mounting section 19, which rest in the circumferential direction and optionally also in the front surface orthogonally thereto on the platform 11. The mounting section 19, hence, has an internal diameter, which essentially corresponds to the external diameter da of the platform 11 of the multi-function syringe 6.

In order to non-releasably connect the bladder syringe attachment 14 to the platform 11, there may be provided several possible connections. The bladder syringe attachment 14 may, for example, be attached at the platform 11 by way of ultra-sound welding. Alternatively, the attachment may also be realized by laser welding or bonding. In particular in a welding process it is preferred that the platform 11 and the bladder syringe attachment 14 are made from the same material, e.g., a polypropylene random copolymer such as PP Purell RP373R.

The FIGS. 7 and 8 each show an embodiment, in which the platform 11 is provided about the circumference with a rib 20. The rib 20 may be formed by a ridge completely extending about the circumference or by several knobs. Complementarily thereto, the bladder syringe attachment 14 has at the internal circumference a groove 21, which corresponds to the shape of the rib 20 in a mirror-inverted way. Alternatively, the platform 11 may also have a groove, and the bladder syringe attachment 14 may have a rib.

The bladder syringe attachment 14 may, hence, be applied onto the platform 11, without any further attachment processes such as welding or bonding having to be carried out. The embodiment variants of FIG. 7 and FIG. 5, however, could also be combined such that the platform 11 and the bladder syringe attachment 14 are connected, on the one hand, by a connection notch/groove and, on the other hand, by a welding connection and/or bonding connection.

FIG. 9 shows the multi-function syringe 6 as a multi-closure syringe 13 having a cap 12 applied thereon. FIG. 10 shows in detail that the cap has a smaller length than the syringe outlet 8 such that the closure by means of the cap 12 does not come into contact with the platform 11. The cap 12 and the syringe outlet 8 are complementarily configured. In addition, a tapering angle of the syringe outlet 8 essentially corresponds to a tapering angle within the cap 12.

1. A multi-function syringe, comprising a syringe barrel and a syringe plunger axially displaceable within the syringe barrel, whereby there is formed a receiving volume enclosed by the syringe barrel and the syringe plunger,
wherein the syringe barrel has a syringe outlet integrally formed thereon for discharging a fluid present in the receiving volume,
wherein the syringe barrel has a cylindrical platform at the transition to the syringe outlet, which has a smaller diameter than a body of the syringe barrel and a larger diameter than the syringe outlet, wherein a bladder syringe attachment having a break-off tip is mountable at the syringe outlet for using the multi-function syringe as a bladder syringe.

2. A multi-function syringe according to claim 1, wherein the syringe outlet has a length and a tip diameter, which deviate from the standard dimensions of a Luer cone standardized in the ISO-80369-7.

3. A multi-function syringe according to claim 1, wherein the syringe outlet has a length and a tip diameter, which correspond to the standard dimensions of a Luer cone standardized in the ISO-80369-7.

4. A multi-function syringe according to claim 1, wherein the syringe barrel is an integrally manufactured injection-moulded part.

5. A multi-function syringe according to claim 1, wherein the platform has at its external circumference a rib extending at least in part around the circumference for attaching the bladder syringe attachment.

6. A bladder syringe, comprising a multi-function syringe according to claim 1 and the bladder syringe attachment with break-off tip, wherein the bladder syringe attachment has a mounting section facing away from the break-off tip and having an internal diameter, which essentially corresponds to the external diameter of the platform of the multi-function syringe.

7. A bladder syringe according to claim 6, wherein the mounting section of the bladder syringe attachment is non-releasably mounted on the platform.

8. A bladder syringe according to claim 6, comprising the multi-function syringe according to claim 4, wherein the mounting section of the bladder syringe attachment has a groove at the internal circumference thereof for engagement with the rib of the platform.

9. A bladder syringe according to claim 6, wherein the multi-function syringe and the bladder syringe attachment are made of the same material.

10. A bladder syringe according to claim 6, wherein the mounting section of the bladder syringe attachment has an external diameter, which corresponds to or is smaller than the diameter of the body of the syringe barrel.

11. A bladder syringe according to claim 6, wherein the bladder syringe attachment has an internal profile, which corresponds essentially to the external profile of the syringe outlet, and wherein the external profile of the syringe outlet rests on the internal profile of the bladder syringe attachment when the bladder syringe attachment is mounted on the multi-function syringe.

12. Multi-closure syringe, in particular wound gel syringe, comprising a multi-function syringe according to claim 1 and a cap, which has a smaller length than the syringe outlet and releasably closes the syringe outlet.

13. Kit-of-Parts, comprising the multi-function syringe according to claim 1, the bladder syringe attachment having the break-off tip and a cap,
wherein the bladder syringe attachment has a mounting section facing away from the break-off tip and having an internal diameter, which essentially corresponds to the external diameter of the platform of the multi-function syringe, and
wherein the cap has a smaller length than the syringe outlet and releasably closes the syringe outlet.

14. A multi-function syringe according to claim 2, wherein the length of the syringe outlet is larger than the length of a Luer cone standardized in the ISO-80369-7 and the tip diameter of the syringe outlet is larger than the tip diameter of a Luer cone standardized in the ISO-80369-7.

15. A bladder syringe according to claim 6, wherein the mounting section of the bladder syringe attachment is non-releasably mounted on the platform by ultra-sound welding, laser welding or bonding.
